(19) United States
(12) Patent Application Publication
(10) Pub. No.: US 2007/0027218 A1
(43) Pub. Date: Feb. 1, 2007

(54) Pharmaceutical compositions comprising higher primary alcohols and ezetimibe and process of preparation thereof

(75) Inventors: Rajesh Jain, New Delhi (IN); Kour Chand Jindal, New Delhi (IN); Sukhjeet Singh, New Delhi (IN)

Correspondence Address: LADAS & PARRY, 26 WEST 61ST STREET, NEW YORK, NY, 10023, US

(73) Assignee: PANACEA BIOTEC LTD.

(21) Appl. No.: 11/489,253

(22) Filed: Jul. 19, 2006

Related U.S. Application Data
(63) Continuation-in-part of application No. PCT/IN05/00025, filed on Jan. 19, 2005.

(30) Foreign Application Priority Data
Jan. 20, 2004 (IN) 103/DEL/2004

(51) Int. Cl. A61K 31/045; A61K 47/00

(52) U.S. Cl. 514/724; 514/772

(57) ABSTRACT

A novel pharmaceutical composition comprising a mixture of higher primary aliphatic alcohols from 24 to 39 carbon atoms; at least one another component selected from resins and pigments, hydrocarbons, esters, ketones and aldehydes, and phenolic compounds, and ezetimibe, its salts, analogs or derivatives thereof optionally with pharmaceutically acceptable excipients, and process of preparation of such composition is provided. Also provided are method of treatment and use of such composition for reducing abnormal lipid parameters associated with hyperlipidemia.

### CROSS REFERENCE

This application is a continuation-in-part of International Application No. IN2005/000025 filed on Jan. 19, 2005, which designated the U.S., claims the benefit thereof and incorporates the same by reference.

### FIELD OF THE INVENTION

The present invention relates to novel pharmaceutical compositions comprising a mixture of higher primary aliphatic alcohols from 24 to 39 carbon atoms; at least one another organic component selected from resins and pigments, hydrocarbons, esters, ketones and aldehydes, and phenolic compounds, and ezetimibe, its salts, analogs or derivatives thereof optionally with pharmaceutically acceptable excipients, and process of preparation of such composition. Also described are method of treatment and use of such composition thereof for reducing abnormal lipid parameters associated with hyperlipidemia. Particularly, the present invention relates to compositions and method for lowering total cholesterol and triglycerides (TGs) level or elevating high density lipoprotein cholesterol (HDL-C) level in blood of a mammal.

### BACKGROUND OF THE INVENTION

Elevated serum cholesterol levels (>200 mg/dl) have been indicated as a major risk factor for heart disease, the leading cause of death worldwide. Atherosclerotic vascular diseases, especially coronary heart disease (CHD), are the major cause of morbidity and mortality in middle age and elderly people worldwide (Pyorala et al., 1994; Sans et al., 1997). Thus, primary and secondary prevention of morbidity and death from CHD represents a major healthcare problem.

However, the use of currently available statins and fibrates should be used with caution in special patient population with increased susceptibility to drug-related adverse effects and frequent consumption of several concomitant medications, such as the elderly, patients with active hepatic diseases, etc. Furthermore, these lipid-lowering drugs are associated with adverse effects such as gastrointestinal disturbances, increases in serum transaminases and creatinine kinase, myopathies, headache, cholelithiasis, impairment of fertility, and diminished libido. Due to the fact that cholesterol-lowering drugs must be administered on a long-term basis, there is still need of new effective and well-tolerated hypocholesterolemic agents.

The regulation of whole body cholesterol homeostasis in humans and animals involve the regulation of dietary cholesterol and modulation of cholesterol biosynthesis, bile acid biosynthesis and the catabolism of the cholesterol-containing plasma lipoproteins. The liver is the major organ responsible for cholesterol biosynthesis and catabolism, and for this reason it is a prime determinant of plasma cholesterol levels. The liver is the site of synthesis and secretion of very low density lipoproteins (VLDL) which are subsequently metabolized to low density lipoproteins (LDL) in the circulation. LDL is the predominant cholesterol-carrying lipoproteins in the plasma and an increase in their concentration is correlated with increased atherosclerosis.

Plant derived long-chain aliphatic alcohols have also been documented to reduce serum cholesterol levels in experimental models, and in type II hypercholesterolemic patients. Mixture of higher primary aliphatic alcohols has been employed in the treatment of elevated serum cholesterol levels. In the past few years such mixtures have shown much promise as reported in a number of published human clinical trials. The mechanism of action of such mixtures is not known, but various studies revealed that such mixtures inhibit cholesterol biosynthesis, increase the number of LDL-C receptors thereby decreases serum TC, LDL-C and increase HDL levels (Menendez et al., 1994).

U.S. Pat. No. 5,856,316 discloses a process for obtaining mixture of higher primary aliphatic alcohols from sugarcane wax and their utilization in the treatment of hypercholesterolemia. Such mixture from sugarcane wax comprise a mixture of aliphatic alcohols from 24 to 34 carbon atoms and they were effective hypocholesterolemic agents administered in daily doses from 1 to 100 mg.

Ezetimibe selectively inhibits the intestinal absorption of cholesterol and related phytosterols leading to a reduction of hepatic cholesterol stores and increase in clearance of cholesterol from blood. When intestinal cholesterol absorption is reduced, by whatever means, less cholesterol is delivered to the liver. The consequence of this action is decreased hepatic lipoprotein (VLDL) production and an increase in the hepatic clearance of plasma cholesterol, mostly as LDL. Thus, the net effect of inhibiting intestinal cholesterol absorption is a decrease in plasma cholesterol levels.

A few azetidinones have been reported as being useful in lowering cholesterol and/or in inhibiting the formation of cholesterol-containing lesions in mammalian arterial walls. U.S. Pat. No. 4,983,597 discloses N-sulfonyl-2-azetidinones as antihyper-cholesterolemic agents.

The U.S. Pat. No. 6,498,156 discloses diphenylazetidinone derivatives, process for their preparation, medicaments comprising these compounds and their use as hypolipidemics.

U.S. Pat. No. RE37721 discloses hydroxy-substituted azetidinone compounds useful as hypocholesterolemic agents. Ram et al (1990) disclose ethyl 4-(2-oxoazetidin-4-yl)phenoxy-alkanoates as hypolipidemic agents.

European Publication No. 264,231 discloses 1-substituted-4-phenyl-3-(2-oxo-alkylidene)-2-azetidinones as blood platelet aggregation inhibitors.

European Publication Nos. 199,630 and 337,549 disclose elastase inhibitory substituted azetidinones said to be useful in treating inflammatory conditions resulting in tissue destruction, which are associated with various disease states, e.g. atherosclerosis.

U.S. Pat. No. 5,846,966 discloses combinations of hydroxy-substituted azetidinone compounds and HMG CoA Reductase Inhibitors.

The US Publication No. 20030232796 relates to nanoparticulate compositions comprising particles of at least one mixture of concentrated n-alkyl alcohols or a salt thereof, wherein the particles have an effective average particle size of less than about 2000 nm; and at least one surface stabilizer preferably selected from the group consisting of an anionic surface stabilizer, a cationic surface stabilizer, a zwitterionic surface stabilizer, and an ionic surface stabilizer. The compositions described additionally comprise one or more active agents resulted from the group comprising of cholesterol lowering agents such as ezetimibe; although no disclosure has been made by way of examples for preparing such composition. However such nanoparticulate compositions are difficult to formulate and the particle size of the active agent becomes very crucial for proper bioavailability and primarily becomes a limiting aspect.

The PCT Publication No. WO 0390547 relates to compositions comprising a waxy acid component consisting of at least a waxy acid with 23 to 50 carbon atoms and/or derivatives thereof and 0 to 99.99% by weight of at least a component with serum cholesterol level effecting properties and 0 to 20% by weight of at least a pharmaceutically acceptable formulation aid.

The mechanism of action of mixture of higher primary aliphatic alcohols is not known, but in vitro studies revealed that the mixture of higher primary aliphatic alcohols inhibit cholesterol biosynthesis at a step located in between acetate consumption and mevalonate production. In addition, in vitro studies also showed that such mixtures increase the number of LDL-C receptors (Menendez et al., 1994). This accounts for the ability of the mixture of higher primary aliphatic alcohols not only to decrease total cholesterol, but also to decrease LDL serum levels and increase HDL levels. In vivo studies in correlation with in vitro studies demonstrated that such mixtures inhibited TC and LDL-C induced by atherogenic diet suggesting possible inhibition of cholesterol biosynthesis (Menendez et al., 1996). In addition, administration of such mixtures to diabetic patients significantly reduced TC and LDL-C levels in the blood (Omayda Torres et al., 1995).

Ezetimibe, a diphenylazetidinone derivative that localizes and appears to act at the brush border membrane of the small intestine and selectively inhibits the intestinal absorption of cholesterol and related phytosterols leading top a decrease in the delivery of intestinal cholesterol to the liver. It dose not inhibit cholesterol synthesis in the liver. This causes a reduction of hepatic cholesterol stores and increase in clearance of cholesterol from blood. It reduces TC, LDL-C, Apo B, and TG, and increases HDL-C in patients with hypercholesterolemia.

It can be seen from the scientific literature that there is still a need for development of new drugs or combinations of existing antihyperlipidemic agents with possible additive, potentiating, or synergistic action and a method of administration which would provide a balanced lipid alteration i.e. reductions in TC, LDL-C, TGs, and apolipoprotein a (Lp(a)) as well as increases in HDL-C, with an acceptable safety profile, especially with regards to liver toxicity and effects on glucose metabolism and uric acid levels in hyperlipidemic patients; and which are cost-effective and easier to formulate; but are still beneficial.

### SUMMARY OF THE INVENTION

It is an objective of the present invention to provide novel pharmaceutical composition comprising a mixture of higher primary aliphatic alcohols from 24 to 39 carbon atoms from 2 to 99.9% by weight of the composition; at least one another organic component selected from resins and pigments, hydrocarbons, esters, ketones and aldehydes, and phenolic compounds from 0.1 to 70% by weight of the composition, and ezetimibe, its salts, analogs or derivatives thereof substantially devoid of any waxy acid, optionally with pharmaceutically acceptable excipients from 0 to 99.9% by weight of the composition.

It is an objective of the present invention to provide a process for preparing such composition which comprises of the following steps:

i) isolating the wax,

ii) subjecting the wax to extraction with a liquid organic extractant in which primary aliphatic alcohols and other organic components are soluble,

iii) recovering said soluble mixture from said extractant,

iv) purifying the extract by repeated washing and crystallization,

v) drying the extract and making it into a powder form,

vi) adding ezetimibe, its salts, analogs or derivatives,

vii) optionally adding pharmaceutically acceptable excipients and making it into a suitable dosage form.

It is yet another objective of the present invention to provide a method of reducing serum cholesterol level, and treating hyperlipidemia, which comprises administering a composition comprising a mixture of higher primary aliphatic alcohols from 24 to 39 carbon atoms from 2 to 99.9% by weight of the composition; at least one another organic component selected from resins and pigments, hydrocarbons, esters, ketones and aldehydes, and phenolic compounds from 0.1 to 70% by weight of the composition, and ezetimibe, its salts, analogs or derivatives thereof, substantially devoid of any waxy acid, optionally with pharmaceutically acceptable excipients from 0 to 99.9% by weight of the composition.

The compositions of the present invention have preferably a synergistic effect for reducing serum cholesterol level in mammals.

### DETAILED DESCRIPTION OF THE INVENTION

The present invention relates to novel pharmaceutical composition comprising a mixture of higher primary aliphatic alcohols from 24 to 39 carbon atoms from 2 to 99.9% by weight of the composition; at least one another organic component selected from resins and pigments, hydrocarbons, esters, ketones and aldehydes, and phenolic compounds from 0.1 to 70% by weight of the composition, and ezetimibe, its salts, analogs or derivatives thereof.

The compositions of the present invention are substantially devoid of any waxy acid, optionally with pharmaceutically acceptable excipients from 0 to 99.9% by weight of the composition.

The mixture of higher primary aliphatic alcohols in the present invention are selected from but not limited to a group comprising 1-tetracosanol, 1-hexacosanol, 1-heptacosanol, 1-octacosanol, 1-nonacosanol, 1-tetratriacontanol, 1-triacontanol, 1-hexacontanol, eicosanol, 1-hexacosanol, 1-tetracosanol, 1-dotriacontanol, 1-tetracontanol, and the like. Preferably the mixture of higher primary aliphatic alcohols comprises 1-tetracosanol, 1-hexacosanol, 1-heptacosanol, 1-octacosanol, and 1-triacontanol.

In a further embodiment, the present invention provides a composition, wherein the mixture of higher primary aliphatic alcohols from 24 to 39 carbon atoms comprising 1-tetracosanol, 1-hexacosanol, 1-heptacosanol, 1-octacosanol, and 1-triacontanol are present as at least 40% by weight of the composition.

In a further embodiment, the present invention provides a composition, wherein the ratio of the mixture of higher primary aliphatic alcohols and ezetimibe, its salts, analogs or derivatives thereof is from 20:1 to 1:20.

In another embodiment of the present invention, the mixture of higher primary aliphatic alcohols from 24 to 39 carbon atoms and the other organic component(s) selected from resins and pigments, hydrocarbons, esters, ketones and aldehydes, and phenolic compounds comprises of the following:

| | |
|---|---|
| 1-tetracosanol | 0.0-2.0% |
| 1-hexacosanol | 0.2-2.0% |
| 1-heptacosanol | 0.0-1.0% |
| 1-octacosanol | 30.0-40.0% |
| 1-triacontanol | 6.0-9.5% |
| Resins and pigments | 5.0-10.0% |
| Hydrocarbons | 1.0-10.0% |
| Esters | 1.0-10.0% |
| Ketones and Aldehydes | 1.0-10.0% |
| Phenolic compounds | 0.0-5.0% |

In a still further embodiment of the present invention, the mixture of higher primary aliphatic alcohols from 24 to 39 carbon atoms and the other organic component(s) selected from resins and pigments, hydrocarbons, esters, ketones and aldehydes, phytosterols, and phenolic compounds comprises of the following:

| | |
|---|---|
| 1-tetracosanol | 0.0-2.0% |
| 1-hexacosanol | 0.2-2.0% |
| 1-heptacosanol | 0.0-1.0% |
| 1-octacosanol | 30.0-40.0% |
| 1-triacontanol | 6.0-9.5% |
| Phytosterols | 0.1-1.0% |
| Resins and pigments | 5.0-10.0% |
| Hydrocarbons | 1.0-10.0% |
| Esters | 1.0-10.0% |
| Ketones and Aldehydes | 1.0-10.0% |
| Phenolic compounds | 0.0-5.0% |

The mixture of high-molecular weight aliphatic alcohols of the present invention occur naturally in wax form and are characterized by fatty alcohol chains ranging from 20 to 39 carbon atoms in length. The major components of such mixture are the aliphatic alcohols 1-octacosanol and 1-triacontanol, and the component includes 1-tetracosanol, 1-hexacosanol, 1-heptacosanol, 1-octacosanol, 1-nonacosanol, 1-tetratriacontanol, 1-triacontanol, 1-hexacontanol, eicosanol, 1-hexacosanol, 1-tetracosanol, 1-dotriacontanol, 1-tetracontanol, and the like; and other organic components such as resins and pigments, hydrocarbons, esters, ketones and aldehydes, phytosterols, phenolic compounds, and the like. Such mixture of high-molecular weight aliphatic alcohols and other organic components of the present invention are preferably isolated from a number of different sources, including sugar cane wax, beeswax, and rice bran wax, more preferably sugar cane wax. It should be understood, however, that the invention is not limited in this regard and that such mixture of high-molecular weight aliphatic alcohols commonly available from other naturally occurring and synthetic sources may be utilized.

In an embodiment, the present invention employs ezetimibe or a compound other than ezetimibe itself that the body metabolizes into ezetimibe, thus producing the same effect as described herein. The other compounds include N-sulfonyl-2-azetidinones, diphenylazetidinone derivatives, hydroxy-substituted azetidinone compounds, ethyl 4-(2-oxoazetidin-4-yl) phenoxy-alkanoates, 1-substituted-4-phenyl-3-(2-oxo-alkylidene)-2-azetidinones or the like, and their analogs or salts thereof. Each such compound will be collectively referred to herein by “ezetimibe.”

The mixture of higher primary aliphatic alcohols and ezetimibe lower serum cholesterol levels by two independent and unrelated mechanisms of action. Interestingly, when the mixture of higher primary aliphatic alcohols and ezetimibe combined showed a significant synergistic effect. The mixture of higher primary aliphatic alcohols inhibit a step located in between acetate consumption and mevalonate production whereas ezetimibe selectively inhibits intestinal cholesterol absorption thereby decreases cholesterol available in the liver. Moreover, the mixture of higher primary aliphatic alcohols increase the number of LDL-C receptors in liver thereby reduces LDL-C levels.

Both the compounds when used alone decrease TGs, VLDL, apoB, and increases HDL-C. Thus, the combination of both these agents into a single composition provides a more effective treatment for elevated serum cholesterol than would be expected from the additive effect of both compounds given separately.

In an embodiment, the present invention provides pharmaceutical compositions suitable for lowering LDL-C and TGs level or elevating HDL-C level in blood of a mammal or both, by incorporating a combination of the mixture of high-molecular weight aliphatic alcohols, and at least one another organic component selected from resins and pigments, hydrocarbons, esters, ketones and aldehydes, and phenolic compounds; with ezetimibe, its salts, analogs or derivatives thereof into some suitable pharmaceutical forms such as tablets or capsules or both which may also comprise a pharmaceutically acceptable excipient(s) such as coloring agent, antioxidant, binder, stabilizer, and the like.

The present invention provides a process for preparation of a fixed dose combination comprising of the mixture of high-molecular weight aliphatic alcohols, and at least one another organic component selected from resins and pigments, hydrocarbons, esters, ketones and aldehydes, and phenolic compounds; with ezetimibe, its salts, analogs or derivatives thereof optionally with pharmaceutically acceptable excipients, which can be formulated as oral dosage forms such as tablets, pills, capsules, gels, finely divided powders, dispersions, suspensions, solutions, emulsions, etc; pulmonary and nasal dosage form such as sprays, aerosols, etc.; topical dosage forms such as gels, ointments, creams, etc; parenteral dosage forms; controlled release formulations; fast melt formulations, lyophilized formulations, delayed release formulations, sustained release, extended release formulations, pulsatile release formulations, and mixed immediate release and controlled release formulations. The compositions of the present invention can be formulated for administration by the route selected from the group consisting of oral, pulmonary, rectal, colonic, parenteral, local, buccal, nasal, and topical.

In an embodiment of the present invention, the compositions can be preferably incorporated into compositions in the form of capsules. These capsules may also comprise pharmaceutically acceptable excipients such as diluent, antioxidant, coloring agent, stabilizer, and the like. Composition can also be provided in the form of tablets comprising combination of the mixture of high-molecular weight aliphatic alcohols, and at least one another organic component selected from resins and pigments, hydrocarbons, esters, ketones and aldehydes, and phenolic compounds with ezetimibe, its salts, analogs or derivatives thereof which may also comprise excipients such as diluent, coloring agent, antioxidant, binder, stabilizer, and the like.

In an embodiment of the present invention, the composition as tablets/capsules or any other suitable pharmaceutical form are meant for lowering LDL-C level or elevating HDL-C level in mammals.

In an embodiment of the present invention, the ratio of the mixture of higher primary aliphatic alcohols or esters thereof and ezetimibe, its salts, analogs or derivatives thereof is from 20:1 to 1:20.

In a further embodiment, the composition comprising a combination of a mixture of higher primary aliphatic alcohols from 24 to 39 carbon atoms comprising 1-tetracosanol, 1-hexacosanol, 1-heptacosanol, 1-octacosanol, and 1-triacontanol; phytosterols; resins and pigments; hydrocarbons; esters; ketones and aldehydes; and phenolic compounds with ezetimibe, its salts, analogs or derivatives thereof, optionally comprises pharmaceutically acceptable excipients.

In a further embodiment, the pharmaceutically acceptable excipients are selected from but not limited to a group comprising diluents, disintegrants, fillers, bulking agents, vehicles, pH adjusting agents, stabilizers, anti-oxidants, binders, buffers, lubricants, antiadherants, coating agents, preservatives, emulsifiers, suspending agents, release controlling agents, polymers, colorants, flavoring agents, plasticizers, solvents, preservatives, glidants, chelating agents and the like; used either alone or in combination thereof.

In the present invention, the diluent is selected from but not limited to a group comprising lactose, cellulose, microcrystalline cellulose, mannitol, dicalcium phosphate, pregelatinized starch, and the like, used either alone or in combination thereof.

In the present invention, the binder is selected from but not limited to a group comprising polyvinylpyrrolidone, cellulose derivatives such as hydroxypropyl methylcellulose, methacrylic acid polymers, acrylic acid polymers, and the like.

The release controlling agents and/or polymers of the present invention comprising of at least one release controlling polymer is selected from but not limited to a group comprising polyvinylpyrrolidone/polyvinylacetate copolymer (Kollidon® SR), methacrylic acid polymers, acrylic acid polymers, cellulose derivative, and the like. The methacrylic acid polymer is selected from a group comprising but not limited to Eudragit® (Degussa) such as Ammonio Methacrylate Copolymer type A USP (Eudragit® RL), Ammonio Methacrylate Copolymer type B USP (Eudragit® RS), Eudragit® RSPO, Eudragit® RLPO, and Eudragit® RS30D.

In an embodiment, the lubricant(s) used in the present invention are selected from, but not limited to a group comprising of stearic acid, magnesium stearate, zinc stearate, glyceryl behenate, cetostearyl alcohol, hydrogenated vegetable oil, and the like used either alone or in combination thereof.

In a further embodiment, the pharmaceutically acceptable excipients are present in about 0.5-80.0% by weight of the composition.

In a further embodiment of the present invention, a process for preparing a composition which comprises of the following steps:
i) isolating the wax,
ii) subjecting the wax to extraction with a liquid organic extractant in which primary aliphatic alcohols and other organic components are soluble,
iii) recovering said soluble mixture from said extractant,
iv) purifying the extract by repeated washing and crystallization,
v) drying the extract at temperature preferably below 70° C. and making it into a powder form,
vi) adding ezetimibe, its salts, analogs or derivatives,
vii) optionally adding pharmaceutically acceptable excipients and making it into a suitable dosage form.

The wax is preferably isolated from a number of different sources, including sugar cane wax, beeswax, and rice bran wax, more preferably sugar cane wax.

The liquid organic extractant of the present invention are selected from but not limited to a group comprising hexane, heptane, petroleum ether, chlorinated hydrocarbons, methanol, ethanol, isopropyl alcohol, ethyl acetate, acetone, ethyl methyl ketone, and the like, or mixtures thereof.

In the said process, the soluble mixture from the said extractant is recovered by distillation, with or without the application of vacuum.

The extract is purified preferably by repeated washing and crystallization. The solvents used for washing are selected from but not limited to hexane, heptane, petroleum ether, methanol, ethanol, isopropyl alcohol, ethyl acetate, acetone, ethyl methyl ketone, and the like, or mixtures thereof and the solvents for crystallization are selected from but not limited to hexane, heptane, petroleum ether, chlorinated hydrocarbons, methanol, ethanol, isopropyl alcohol, ethyl acetate, acetone, ethyl methyl ketone, toluene, and the like, or mixtures thereof.

The extract is dried by subjecting it to hot air oven, or by a Fluid bed drier, preferably at temperature below 70° C.

The present invention also provides a method of reducing serum cholesterol level, and treating hyperlipidemia, which comprises administering a composition comprising a mixture of higher primary aliphatic alcohols from 24 to 39 carbon atoms from 2 to 99.9% by weight of the composition; at least one another organic component selected from resins and pigments, hydrocarbons, esters, ketones and aldehydes, and phenolic compounds from 0.1 to 70% by weight of the composition, and ezetimibe, its salts, analogs or derivatives thereof, substantially devoid of any waxy acid, optionally with excipients from 0 to 99.9% by weight of the composition. The compositions of the present invention have preferably a synergistic effect for reducing serum cholesterol level, and treating hyperlipidemia, particularly in mammals.

The ability of the mixture of higher primary aliphatic alcohols to inhibit cholesterol synthesis and of ezetimibe, its salts, analogs or derivatives thereof to decrease total cholesterol (TC), low density lipoprotein cholesterol (LDL-C), TGs, and lipoprotein (a) (Lp(a)) while increasing HDL-C; when combined in the present invention results in preferably a synergistic effect in lowering serum cholesterol.

In an embodiment, the compositions for lowering LDL-C level or elevating HDL-C level in blood of a mammal or both, comprise a mixture of higher primary aliphatic alcohols, and at least one another organic component selected from resins and pigments, hydrocarbons, esters, ketones and aldehydes, and phenolic compounds; with ezetimibe, its salts, analogs or derivatives thereof, and a method for lowering LDL-C and/or TGs level or elevating HDL-C level in blood of a mammal or both, comprises orally administering to said mammal, such compositions.

In an aspect of the present invention, the lipid lowering compositions comprising a mixture of higher primary aliphatic alcohols; at least one another organic component selected from resins and pigments, hydrocarbons, esters, ketones and aldehydes, and phenolic compounds; and ezetimibe, its salts, analogs or derivatives thereof is associated with a reduction in the dose of ezetimibe, its salts, analogs or derivatives thereof and increased patient compliance.

In the present invention, the mixture of higher primary aliphatic alcohols from 24 to 39 carbon atoms; and other organic components such as resins and pigments, hydrocarbons, esters, ketones and aldehydes, and phenolic compounds; is denoted as ‘Extract-A’.

### Determination of Biological Activity

### Casein-Starch-Induced Hypercholesterolemia in Rabbits

The observed unexpected synergistic lipid lowering effect of combination of Extract-A as described herein and ezetimibe is evidenced by the test conducted in rabbits. Rabbits of either sex were procured from Central Animal House facility; Panacea Biotec Ltd., India. Animals weighing 1.5-2.0 g at the time of testing were used throughout. All animals were dosed sequentially by the oral route with Extract-A and/or ezetimibe suspended in 0.5% of carboxymethyl cellulose (CMC). A dosing volume of 2 ml/kg was used for each sequential suspension.

The fasting serum lipid profile (TC, TGs, LDL-C, HDL-C) was estimated before initiation of the experiment. Total study duration was 90 days. Hypercholesterolemia was induced by feeding rabbits with wheat casein-starch diet (g/kg) containing wheat flour 333, cellulose 300, casein 270, water 20, maize oil 10, and mineral mixture (Kroon et al., 1982) for 8 weeks. Feed consumption was restricted to 100 g/day per animal. The cholesterol level was estimated every 15 days. After 60 days animals with total cholesterol level>150 mg/dl were randomized to treatment (n=6/group). Thereafter, various doses of Extract-A and/or ezetimibe were administered for another 60 days during which animals were fed with casein-starch diet. Blood samples were collected from fasted rabbits and analyzed for any alteration in serum lipid profile after 60 days of test compound(s) administration.

All the data are expressed as mean±S.E.M. (Standard Error of Mean). Student t-test was used to compare the lipid parameters between animals fed with standard and hypercholesterolemic diet. The difference between various drug treated groups was analyzed by ANOVA followed by Dunnett's test. A value of P<0.05 was considered as statistically significant.

Rabbits fed with hypercholesterolemic diet for 60 days produced an increase in serum total cholesterol (TC) and LDL-C level in time dependent manner. Extract-A (100 and 200 mg/kg, p.o.) and ezetimibe (5 and 10 mg/kg, p.o.) reversed TC and LDL-C levels in comparison to hypercholesterolemic control rabbits. Lower doses of Extract-A (100 and 200 mg/kg) and ezetimibe (5 and 10 mg/kg) administered in combination, significantly potentiated the reduction in TC and LDL-C levels. There was no significant change in the body weight of casein-starch fed diet in comparison to initial body weight.

The data for the study is presented in Tables 1 & 2, and shown diagrammatically in FIGS. 1 & 2.

TABLE 1
Effect of Extract-A and/or ezetimibe on serum total cholesterol level in rabbits

| Treatment | 0 | 15 | 30 | 60 | 75 | 90 | 105 | 120 |
|---|---|---|---|---|---|---|---|---|
| CNT | 39.83 ± 2.79 | 119.83 ± 3.87 | 171.16 ± 7.88 | 231.83 ± 7.72 | 270 ± 6.55 | 290.00 ± 9.66 | 325.00 ± 8.07 | 350.00 ± 5.76 |
| Extract-A | 41.00 ± 2.3 | 102.166 ± 3.04 | 161.5 ± 6.42 | 227.6 ± 5.92 | 226.1 ± 6.19* | 234.1 ± 7.23* | 215.83 ± 10.37* | 205.5 ± 15.3* |
| 100 | | | | | | | | |
| Extract-A | 38.16 ± 2.5 | 101.0 ± 2.03 | 163.83 ± 11.84 | 227.83 ± 3.78 | 206.6 ± 5.34* | 192.1 ± 3.08* | 186.67 ± 4.99* | 174.83 ± 4.39* |
| 200 | | | | | | | | |
| Ez-5 | 52.50 ± 3.59 | 103.50 ± 2.23 | 169.83 ± 6.16 | 219.83 ± 7.5 | 236.50 ± 3.47* | 222.83 ± 2.77* | 201.00 ± 12.6* | 181.50 ± 8.55* |
| Ez-10 | 46.50 ± 2.2 | 106.83 ± 3.38 | 162.50 ± 11.54 | 234.50 ± 14.24 | 220.33 ± 8.73* | 197.66 ± 5.42* | 172.16 ± 6.58* | 171.50 ± 4.7* |
| Extract-A | 49.5 ± 2.29 | 110.50 ± 5.21 | 146.00 ± 6.53 | 226.67 ± 11.33 | 175.83 ± 3.89^{a} | 159.10 ± 3.41^{a} | 129.66 ± 6.97^{a} | 117.16 ± 47^{a} |
| 100 + Ez-5 | | | | | | | | |
| Extract-A | 41.00 ± 2.3 | 102.16 ± 3.04 | 161.50 ± 6.42 | 212.16 ± 6.31 | 158.60 ± 5.85^{a} | 135.83 ± 3.19^{a} | 102.50 ± 1.9^{a} | 75.33 ± 5.9^{a} |
| 200 + Ez-10 | | | | | | | | |

*P < 0.05 as compared with control (CNT);
^{a}P < 0.05 as compared with Extract-A 100 and 200 mg/kg, p.o., ezetimibe (Ez) 5 and 10 mg/kg, p.o.

TABLE 2
Effect of Extract-A and/or ezetimibe on LDL-C level in rabbits

| Treatment | 0 | 15 | 30 | 60 | 75 | 90 | 105 | 120 |
|---|---|---|---|---|---|---|---|---|
| CNT | 18.10 ± 1.53 | 67.60 ± 5.3 | 130.73 ± 7.08 | 193.23 ± 8.06 | 213.8 ± 7.6 | 242.93 ± 9.39 | 290.17 ± 7.64 | 325.73 ± 7.58 |
| Extract-A 100 | 18.70 ± 3.32 | 78.23 ± 5.59 | 121.10 ± 7.54 | 184.67 ± 8.46 | 181.47 ± 6.8* | 188.03 ± 7.46* | 169.37 ± 7.71* | 153.07 ± 8.62* |
| Extract-A 200 | 22.80 ± 5.4 | 76.30 ± 13.09 | 126.07 ± 3.31 | 186.9 ± 3.31 | 160.17 ± 3.79* | 147.57 ± 3.86* | 140.73 ± 6.3* | 123.90 ± 4.09* |
| Ez-5 | 22.40 ± 8.81 | 76.13 ± 3.11 | 131.53 ± 6.89 | 174.3 ± 8.02 | 194.07 ± 4.29* | 179.47 ± 3.74* | 156.47 ± 12.92* | 129.17 ± 9.02* |
| Ez-10 | 23.70 ± 7.98 | 72.70 ± 3.92 | 125.07 ± 10.94 | 197.03 ± 13.55 | 179.83 ± 9.83* | 152.80 ± 6.17* | 126.60 ± 6.85* | 124.63 ± 6.55* |
| Extract-A | 21.70 ± 5.45 | 77.00 ± 3.67 | 108.40 ± 5.7 | 182.9 ± 10.83 | 133.67 ± 4.33^{a} | 112.17 ± 4.44^{a} | 76.00 ± 7.12^{a} | 66.37 ± 8.43^{a} |
| 100 + Ez-5 | | | | | | | | |
| Extract-A | 24.87 ± 6.28 | 70.03 ± 4.55 | 119.63 ± 7.03 | 185.67 ± 10.45 | 117.23 ± 4.04^{a} | 102.17 ± 6.71^{a} | 73.63 ± 7.09^{a} | 52.67 ± 9.95^{a} |
| 200 + Ez-10 | | | | | | | | |

*P < 0.05 as compared with control (CNT);
^{a}P < 0.05 as compared with Extract-A (100 and 200 mg/kg, p.o.}, ezetimibe (Ez) (5 and 10 mg/kg, p.o).

### DESCRIPTION OF FIGURES

FIG. 1: Effect of Extract-A and/or ezetimibe on serum total cholesterol level in rabbits

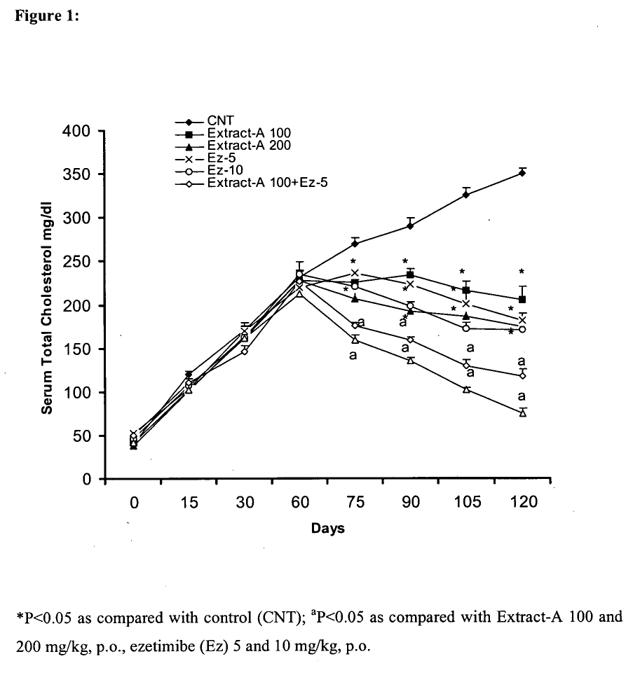

FIG. 2: Effect of Extract-A and/or ezetimibe on LDL-C level in rabbits

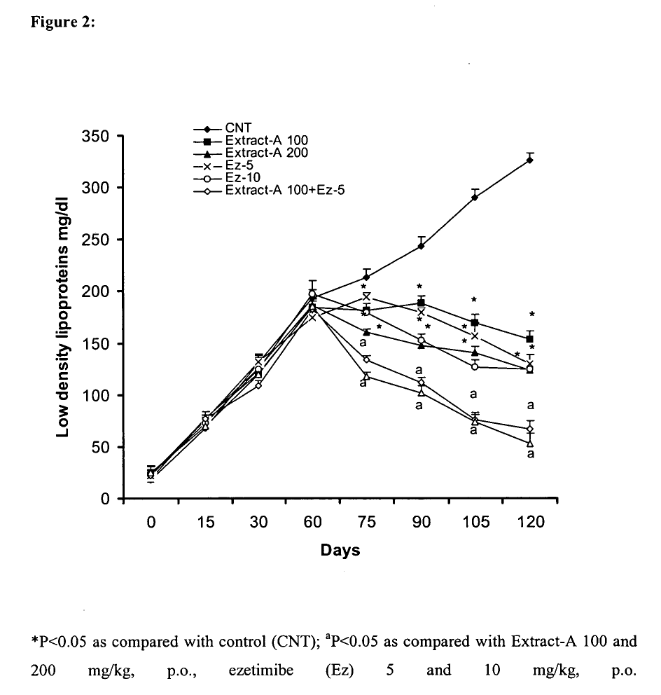

The examples given below serve to illustrate embodiments of the present invention. However they do not intend to limit the scope of present invention.

### EXAMPLES

### Preparation of Extract

### Example 1

4 kg of air-dried Sugar mill Filter cake (or Press Mud) obtained as a byproduct during sugar manufacture from sugarcane was pulverized and extracted four times by boiling with 20 L of dichloroethane each time. The dichloroethane extract was filtered and the solvent was distilled off to get a dark green residue (400 g). The residue was extracted with 4 L of boiling methanol 3 times and the extract was filtered to remove the pitch while still hot (temperature above 50° C.). The filtered extract was distilled to remove methanol till a green residue (200 g) is obtained. The residue was dissolved in 2 L of boiling ethyl methyl ketone and set aside for crystallization. After complete crystallization the solvent is filtered, concentrated to half its volume by distillation and set aside for crystallization of the second crop. Both the crops were pooled and washed with cold hexane. The crystallization and washing procedures were repeated once more. The final washed crystals were dried under a current of air at a temperature not exceeding 70° C. The resultant creamish yellow lumps were pulverized to a fine powder (50 g).

### Example 2

Beeswax obtained after extraction of honey from honeycomb was dried and pulverized and extracted four times by boiling with of ethyl alcohol each time. The alcoholic extract was filtered and the solvent was distilled off to get a residue. The residue was extracted with boiling methanol 3 times and the extract was filtered to remove the pitch while still hot (temperature above 50° C.). The filtered extract was distilled to remove methanol till a green residue is obtained. The residue was dissolved in boiling ethyl acetate and set aside for crystallization. After complete crystallization the solvent is filtered, concentrated to half its volume by distillation and set aside for crystallization of the second crop. Both the crops were pooled and washed with cold hexane. The crystallization and washing procedures were repeated once more. The final washed crystals were dried under a current of air at a temperature not exceeding 70° C. The resultant lumps were pulverized to a fine powder.

### Example 3

4 kg of air-dried Sugar mill Filter cake (or Press Mud) was pulverized and extracted four times by boiling with 20 L of hexane each time. The hexane extract was filtered and the solvent was distilled off to get a dark green residue (350 g). The residue was extracted with 3.5 L of boiling methanol 3 times and the extract was filtered to remove the pitch while still hot (temperature above 50° C.). The filtered extract was distilled to remove methanol till a green residue (200 g) is obtained. The residue was dissolved in 2 L of boiling acetone and set aside for crystallization. After complete crystallization the solvent is filtered, concentrated to half its volume by distillation and set aside for crystallization of the second crop. Both the crops were pooled and washed with cold hexane. The crystallization and washing procedures were repeated once more. The final washed crystals were dried under a current of air at a temperature not exceeding 70° C. The resultant creamish yellow lumps were pulverized to a fine powder (45 g).

### Example 4

10 kg of air-dried Sugar mill Filter cake (or Press Mud) was pulverized and extracted four times by boiling with 50 L of methanol each time. The methanol extract was filtered and the solvent was distilled off to get a dark green residue (650 g). The residue was extracted with 6.5 L of boiling methanol 3 times and the extract was filtered to remove the pitch while still hot (temperature above 50° C.). The filtered extract was distilled to remove methanol till a green residue (500 g) is obtained. The residue was dissolved in 2 L of boiling ethyl acetate and set aside for crystallization. After complete crystallization the solvent is filtered, concentrated to half its volume by distillation and set aside for crystallization of the second crop. Both the crops were pooled and washed with cold hexane. The crystallization and washing procedures were repeated once more. The final washed crystals were dried under a current of air at a temperature not exceeding 70° C. The resultant creamish yellow lumps were pulverized to a fine powder (102 g).

### Preparation of Compositions

### Example 5

### Capsule

| Ingredient | mg/capsule |
|---|---|
| i) Extract-A | 80.0 |
| ii) Ezetimibe | 5.0 |
| iii) Microcrystalline cellulose | 200.8 |
| iv) Mannitol | 72.0 |
| v) Talc | 3.2 |
| vi) Sodium starch glycollate | 12.0 |
| vii) Colloidal silicon dioxide | 12.0 |

Procedure:

1) Extract-A, ezetimibe, microcrystalline cellulose and mannitol are sifted and mixed together.

2) Talc, sodium starch glycollate and colloidal silicon dioxide are passed through fine sieves individually and then mixed together.

3) The materials of step (1) and (2) are mixed and filled into empty hard gelatin capsules

### Example 6

### Uncoated Tablet

| Ingredient | mg/tablet |
|---|---|
| i) Extract-A | 80.0 |
| ii) Ezetimibe | 10.0 |
| iii) Microcrystalline cellulose | 120.0 |
| iv) Mannitol | 80.0 |
| v) Croscarmellose sodium | 20.0 |
| vi) Lactose | 66.0 |
| vii) Talc | 4.0 |
| viii) Colloidal silicon dioxide | 10.0 |

Procedure:

1) Extract-A, ezetimibe, microcrystalline cellulose, mannitol, part of croscarmellose sodium and lactose are sifted and mixed together.

2) The material of step (1) is compacted.

3) The compacts of step (2) are passed through sieve and mixed.

4) Talc, colloidal silicon dioxide and remaining part of croscarmellose sodium are passed through fine sieve and mixed together.

5) The material of step (3) is mixed with material of step (4).

6) The material of step (5) is compressed into tablets.

### Example 7

### Film-Coated Tablet

| Ingredient | mg/tablet |
|---|---|
| Core tablet composition | |
| i) Extract-A | 40.0 |
| ii) Ezetimibe | 10.0 |
| iii) Microcrystalline cellulose | 120.0 |
| iv) Mannitol | 80.0 |
| v) Croscarmellose sodium | 10.0 |
| vi) Lactose | 66.0 |
| vii) Talc | 4.0 |
| viii) Colloidal silicon dioxide | 10.0 |
| ix) Croscarmellose sodium | 10.0 |
| Film coating composition | |
| x) Hydroxypropyl methylcellulose (E-15) | 12.0 |
| xi) Polyethylene glycol 400 (PEG 400) | 2.4 |
| xii) Iron oxide red | 0.75 |
| xiii) Iron oxide yellow | 0.50 |
| xiv) Titanium dioxide | 0.25 |
| xv) Isopropyl alcohol | q.s. (lost in processing) |
| xvi) Dichloromethane | q.s. (lost in processing) |

Procedure:

1) Extract-A, ezetimibe, microcrystalline cellulose, mannitol, croscarmellose sodium and lactose are sifted and mixed together.

2) The material of step (1) is compacted.

3) The compacts of step (2) are passed through sieve and mixed.

4) Talc, colloidal silicon dioxide and croscarmellose sodium are passed through fine sieve and mixed together.

5) The material of step (3) is mixed with material of step (4).

6) The material of step (5) is compressed into tablets.

7) Hydroxypropyl methylcellulose is dispersed in a mixture of isopropyl alcohol and dichloromethane with continuous mixing in homogenizer.

8) PEG 400 is added to the above solution of step (7) and mixed.

9) Iron oxide red, iron oxide yellow and titanium dioxide are passed through fine sieve and mixed.

10) The material of step (9) is added to material of step (8) and mixed for 30 minutes.

11) The core tablets are charged into the coating pan and coated with the coating solution of step (10) till an average tablet weight gain of ˜2-3% is achieved.

We claim:

1. A composition comprising a mixture of higher primary aliphatic alcohols from 24 to 39 carbon atoms from 2 to 99.9% by weight of the composition; at least one other organic component selected from resins, pigments, hydrocarbons, esters, ketones, aldehydes, and phenolic compounds from 0.1 to 70% by weight of the composition, and ezetimibe, or salt, analog or derivative thereof; optionally with excipients from 0 to 80% by weight of the composition; wherein the composition is substantially devoid of any waxy acid.

2. The composition according to claim 1, wherein the mixture of higher primary aliphatic alcohols comprises 1-tetracosanol, 1-hexacosanol, 1-heptacosanol, 1-octacosanol, and 1-triacontanol.

3. The composition according to claim 1, wherein the mixture of higher primary aliphatic alcohols from 24 to 39 carbon atoms comprises 1-tetracosanol, 1-hexacosanol, 1-heptacosanol, 1-octacosanol, and 1-triacontanol and are present as at least 40% by weight of the composition.

4. The composition according to claim 1, wherein the ratio of the mixture of higher primary aliphatic alcohols and ezetimibe or, a salt, analog or derivative thereof is from 20:1 to 1:20 weight/ weight.

5. The composition according to claim 1, wherein the pharmaceutically acceptable excipients are selected from the group consisting of diluents, disintegrants, fillers, bulking agents, vehicles, pH adjusting agents, stabilizers, anti-oxidants, binders, buffers, lubricants, antiadherants, coating agents, preservatives, emulsifiers, suspending agents, release controlling agents, polymers, colorants, flavoring agents, plasticizers, solvents, preservatives, glidants, and chelating agents or a mixture thereof.

6. The composition according to claim 1, which is formulated in an oral; pulmonary; nasal; topical; parenteral; controlled release; fast melt; lyophilized; delayed release; sustained release; extended release; pulsatile release; mixed immediate release; or controlled dosage form.

7. The composition according to claim 6, wherein the oral dosage form is selected from the group consisting of a tablet, pill, capsule, gel, powder, dispersion, suspension, solution and emulsion.

8. The composition according to claim 6, wherein the nasal or pulmonary dosage form is a spray or aerosol.

9. The composition according to claim 6, wherein the topical dosage form is selected from the group consisting of a gel, ointment and cream.

10. A process for preparing a composition according to claim 1, which comprises the steps of:
i) isolating a wax,
ii) subjecting the wax to extraction with a liquid organic extractant in which primary aliphatic alcohols and other organic components are soluble,
iii) recovering said soluble mixture as an extract from said extractant,
iv) purifying the extract by repeated washing and crystallization,
v) drying the extract and making it into a powder form,
vi) adding ezetimibe, a salt, analog or derivatives thereof,
vii) optionally adding pharmaceutically acceptable excipients, and making it into a suitable dosage form.

11. The process according to claim 10, wherein the mixture of higher primary aliphatic alcohols comprises 1-tetracosanol, 1-hexacosanol, 1-heptacosanol, 1-octacosanol, and 1-triacontanol.

12. A process according to claim 10, wherein the mixture of higher primary aliphatic alcohols from 24 to 39 carbon atoms comprises 1-tetracosanol, 1-hexacosanol, 1-heptacosanol, 1-octacosanol, and 1-triacontanol are present as at least 40% by weight of the composition.

13. The process according to claim 10, wherein the ratio of the mixture of higher primary aliphatic alcohols and ezetimibe, a salt, analog or derivative thereof is from 20:1 to 1:20 weight/weight.

14. A method of reducing serum cholesterol level, and treating hyperlipidemia, which comprises administering a composition comprising a mixture of higher primary aliphatic alcohols from 24 to 39 carbon atoms from 2 to 99.9% by weight of the composition; at least one another organic component selected from resins, pigments, hydrocarbons, esters, ketones, aldehydes, and phenolic compounds from 0.1 to 70% by weight of the composition, and ezetimibe or, a salt, analog or derivatives thereof; optionally with excipients from 0 to 80% by weight of the composition; wherein the composition is substantially devoid of any waxy acid.
